(19) United States
(12) Patent Application Publication
(10) Pub. No.: US 2011/0059127 A1
(43) Pub. Date: Mar. 10, 2011

(54) USE OF MUTANT HIV-1 PROTEASE OR SIV PROTEASE AS AN ADJUVANT

(75) Inventors: Young Chul Sung, Gyeongbuk (KR); Dong Bin Jin, Gyeongbuk (KR); Kwang Soon Kim, Gyeonggi-do (KR)

(73) Assignee: POSTECH FOUNDATION, Gyeongbuk (KR)

(21) Appl. No.: 12/918,759

(22) PCT Filed: Feb. 22, 2008
(86) PCT No.: PCT/KR08/01067

§ 371 Date: Nov. 15, 2010

(51) Int. Cl. A61K 39/295; A61K 47/42; A61K 39/21; A61K 47/36; A61P 37/04; A61P 31/18; A61P 31/12; A61P 31/14

(52) U.S. Cl. 424/202.1; 424/278.1; 424/208.1

(57) ABSTRACT

The present invention relates to a mutant HIV-1 (Human immunodeficiency virus-1) protease capable of effectively enhancing cell-mediated immune responses to DNA vaccination, and use of a nucleic acid encoding the same as a vaccine adjuvant. The mutant HIV-1 protease according to the present invention has inactivated or attenuated proteolytic activity, while retaining chaperone-like activity. When the mutant HIV-1 protease is used together with a DNA vaccine against the HIV-1 envelope protein or the HPV antigen (E6 or E7), cell-mediated immune responses can be effectively enhanced for the prevention or treatment of AIDS or cervical cancer.

### TECHNICAL FIELD

The present invention relates to the use of a mutant HIV-1 (Human immunodeficiency virus-1) protease or a mutant SIV (Simian immunodeficiency virus) protease as a vaccine adjuvant, in which the mutant has inactivated or attenuated proteolytic activity, while retaining chaperone-like activity of HIV-1 protease or SIV protease.

### BACKGROUND ART

Unlike the known protein vaccines, DNA vaccine is a form of plasmid that contains a gene encoding an antigen of interest. After immunization, DNA vaccine is taken up by a variety of cells including dendritic cells in the body of recipient, and induces transcription of the antigen gene and antigen production. The produced antigens are presented to B and T cells, leading to humoral and cell-mediated immune responses.

DNA vaccines have several advantages over the traditional vaccines. DNA vaccines are able to induce persistent expression of antigen with no apparent harmful side effects on human body, and are more effective for the induction of cell-mediated immune response unlike the known protein vaccines. In addition, since DNA vaccines are very easy to produce and stable at room temperature, they do not require a cold-chain, which is needed during storage and distribution of the traditional vaccines (Gurunathan, S., et al., Ann. Rev. Immunol., 2000, 18:927-974).

However, DNA vaccines have limitations such as the low immunogenicity in large animals like monkey, chimpanzee, and human, whereas they are able to induce relatively strong humoral and cell-mediated immune responses in small animals such as mouse.

To overcome this drawback, immune regulatory factors such as cytokines and chemokines, apoptosis-inducing molecules capable of enhancing antigen targeting to dendritic cells, and chaperone genes have been used as an adjuvant for DNA vaccine (Donnelly, J. J., et al., J. Immunol., 2005, 175:633-639).

Among them, genes encoding pro-apoptotic molecules such as caspase-2 or caspase-3 can induce the apoptosis of DNA vaccine transfected cells, which facilitates antigen uptake and processing by dendritic cells. Thus, cell-mediated immune response can be more effectively induced in DNA vaccine adjuvanted with pro-apoptotic genes. (Sasaki, S., et al., Nat. Biotechnol., 2001, 19:543-547, Kojima, Y., et al., Vaccine, 2007, 25:438-445). There is another reason that cellular antigens are highly immunogenic than soluble ones (Li, M., et al., J. Immunol., 2001, 166:6099-6103).

it was also suggested that chaperones such as HSP-70 (heat shock protein-70) bind with antigen peptides that are derived from the antigen expressed by DNA vaccine, so as to protect the antigen peptides from peptidase, and to enhance antigen targeting to dendritic cells through receptors expressed by dendritic cells, such as CD91 and TLR4 (Srivastava, P. Annu. Rev. Immunol., 2002, 20: 395-425, Binder, R. J., et al., Nat. Immunol., 2005, 6:593-599, Floto, R. A., et al, Science, 2006, 314:454-458).

HIV-1 protease is a typical aspartic protease, and is involved in proteolytic processing of HIV-1 polyproteins such as gag and pol, and is needed for HIV-1 production (Oroszlan, S., et al., Curr. Top. Microbiol. Immunol., 1990, 157:153-185). In addition, HIV-1 protease processes cytoskeletal proteins, the anti-apoptotic protein Bcl-2, and procaspase-8, leading to degradation thereof (Strack, P. R., et al., Proc. Natl. Acad. Sci. USA, 1996, 93:9571-9576, Nie, Z., et al., Cell Death Differ., 2002, 9:1172-1184, Shoeman, R. L., et al., Proc. Natl. Acad. Sci. USA, 1990, 87:6336-6340).

One of the unique functions of HIV-1 protease is cytotoxicity, which can be induced by HIV-1 infection or transfer of HIV-1 protease gene into cells. It was suggested that the cytotoxic effect of HIV-1 protease is controlled by its proteolytic activity. it was also demonstrated that cell death can be suppressed by genetic mutation of the enzyme's active site or treatment with protease inhibitors capable of inhibiting the protease activity (Blanco, R., et al., J. Biol. Chem., 2003, 278:1086-1093, Ventoso, I., et al., Antiviral Res., 2005, 66:47-55).

Recent discovery has clarified that HIV-1 protease has a chaperone-like activity of preventing protein misfolding and aggregation in vitro (Hulko, M., et al., Protein Sci., 2007, 16:644-653). This activity requires the substrate binding sites, but removal of the catalytic aspartic acid residue by mutation does not affect the chaperone-like activity.

### DISCLOSURE

### Technical Problem

It is an object of the present invention to provide a method for overcoming the low potency of DNA vaccines to induce T cell immune response.

In particular, the object of the present invention is to provide a mutant HIV-1 protease as an adjuvant of DNA vaccine for enhancement of immune responses, which is accomplished by reducing the inhibitory effect of HIV-1 protease on antigen expression and retaining its chaperone-like activity.

It is another object of the present invention to provide a method for enhancing antigen-specific, cell-mediated immune responses by immunization of antigen-expressing DNA vaccine together with the above adjuvant of DNA vaccine.

### Technical Solution

Therefore, the present inventors have proposed that the mutant HIV-1 protease can be used as an adjuvant to enhance T cell immune responses to DNA vaccination. In detail, immune-enhancing effects such as chaperone activity are maximized by mutations that attenuate or inactivate the proteolytic activity of HIV-1 protease, so as to greatly enhance antigen-specific cell-mediated immune responses to DNA vaccination.

### ADVANTAGEOUS EFFECTS

The mutant HIV-1 protease or mutant SIV protease according to the present invention can be used to enhance cell-mediated immune responses to DNA vaccination, regardless of the type of antigen or the mouse model. Since cell-mediated immune responses are thought to be necessary to prevent or treat HIV, hepatitis B or C and cancer, the mutant protease can be effectively utilized as an adjuvant for DNA vaccines or therapeutic agents for the prevention or treatment of the diseases.

### DESCRIPTION OF DRAWINGS

FIG. 1 shows the construction of the vector expressing a mutant HIV-1 protease;

FIG. 2 shows the results of comparing enzymatic activities between wild-type and mutant HIV-protease by analyzing their proteolytic activity on HIV-1 Gag polyprotein;

FIG. 3 shows the results of enhancing antigen-specific cell-mediated immune response by the co-administration of PRatt or PRina together with HIV-1 (Human Immunodeficiency Virus) Env antigen;

FIG. 4 shows the results of enhancing antigen-specific cell-mediated immune response by the co-administration of PRatt or PRina together with HPV (Human Papillomavirus) 16 E67 antigen;

FIG. 5 shows the results of measuring tumor size (A) and survival rate (B) in mouse challenged with tumor cell line (TC-1) expressing HPV16 E6 and E7 to analyze anti-tumor effects, after injecting PRatt or PRina as a DNA vaccine adjuvant together with a plasmid (E67) expressing HPV (Human Papillomavirus) 16 E6 and E7 antigens.

FIG. 6 is the results showing CD4 T cell immune response to wild type or mutant HIV-1 protease in wild type mouse (A) and immune response to E7 in MHC class II^{-/-} mouse (B) after co-administration of wild type or mutant HIV-1 protease with HPV E67 DNA vaccine.

### BEST MODE

In accordance with one aspect, the present invention relates to a vaccine adjuvant comprising a mutant HIV-1 (Human immunodeficiency virus-1) protease or a mutant SIV (Simian immunodeficiency virus) protease which has inactivated or attenuated proteolytic activity, while retaining chaperone-like activity of HIV-1 protease or SIV protease.

Preferably, the vaccine adjuvant is an expression vector containing gene which codes for the mutant HIV-1 protease or mutant SIV protease. More preferably, the vaccine adjuvant is characterized in that it promotes cell-mediated immune responses to DNA vaccination, when used together with the DNA vaccine containing gene encoding an antigen. However, it will be readily understood by those skilled in the art that the adjuvant according to the present invention may be, but is not limited to, in the form of nucleic acid, and may also exist in the form of polypeptide derived therefrom. In addition, it will be readily understood by those skilled in the art that the adjuvant according to the present invention may be used together with the known protein vaccines, as well as DNA vaccines, as long as it enhances cell-mediated immune responses.

In accordance with the preferred embodiment of the present invention, the “mutant. HIV-1 protease” refers to a mutant HIV-1 protease that has inactivated or attenuated proteolytic activity, while retaining chaperone-like activity, by modification of the enzyme active site, -Asp-Thr-Gly- in the amino acid sequence of wild type HIV-1 protease. In accordance with the specific embodiment, the “mutant HIV-1 protease” refers to a mutant HIV-1 protease that results from amino acid modification including substitution, deletion or insertion at the enzyme active site, -Asp-Thr-Gly- in the amino acid sequence of wild type HIV-1 protease.

In the preferred Example of the present invention, a mutant HIV-1 protease with attenuated or inactivated proteolytic activity was prepared by substituting other amino acids, particularly, serine (Ser) for threonine (Thr) or alanine (Ala) for aspartic acid (Asp) at the enzyme active site -Asp-Thr-Gly- in the amino acid sequence of wild type HIV-1 protease, respectively. It was confirmed that the mutant protease can significantly enhance cell-mediated immune responses to HIV-1 Env antigen and cell-mediated immune responses to HPV (Human Papillomavirus) E6 or E7 antigen.

Meanwhile, in the preferred Example of the present invention, the nucleic acid sequence encoding the mutant HIV-1 protease was codon-optimized to increase its expression in mammalian cells, in particular, human cells. The term “codon optimization”, as used herein, refers to substituting a codon having high preference in some hosts among the codons designating the amino acids upon transcription and translation of DNAs to a protein in a host cell, with a codon having a higher preference, and thus increasing the expression efficiency of the amino acid or protein, encoded by the nucleic acids.

As used herein, “PRwt, PRatt and PRina” refer to the codon-optimized wild type (wt) HIV-1 protease, the codon-optimized mutant HIV-1 protease with attenuated proteolytic activity, and the codon-optimized mutant HIV-1 protease with inactivated proteolytic activity, respectively.

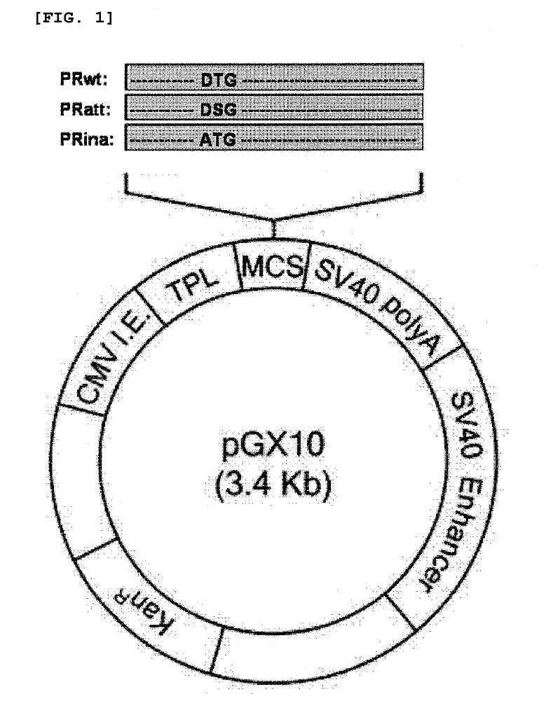

In the preferred Example of the present invention, a plasmid vector was prepared by inserting the codon-optimized gene encoding the mutant HIV-1 protease into the expression vector, preferably pGX10 vector (see FIG. 1) that was developed by the present inventors and disclosed in Korean Patent Application NO. 10-2006-76619 and PCT/KR2006/003181 (see Example 1). However, it will be readily understood by those skilled in the art that the present invention is not limited thereto, and the nucleic acid may be integrated into the well known virus-derived vector to be employed in the design of the viral vector. Examples of the well known virus-derived vector may include vectors derived from adenovirus, lentivirus, retrovirus, adeno-associated virus, vaccinia virus, and alphavirus, but are not limited thereto.

In the present invention, the effect of the mutant HIV-1 protease, of which cytotoxic activity is inhibited by attenuation or inactivation of its proteolytic activity, on the immune responses to the antigen expressed by DNA vaccine, was investigated by comparing to that of the wild type HIV-1 protease with cytotoxic activity. To investigate the effects, the HIV-1 antigen (the cause of AIDS) and the HPV antigen (the cause of cervical cancer) were used, and the antigen-specific, cell-mediated and humoral immune responses, which were induced by the co-immunization of the wild type HIV-1 protease or two mutants thereof with an antigen-expressing DNA vaccine, were evaluated by IFN-g ELISPOT and IgG ELISA, respectively.

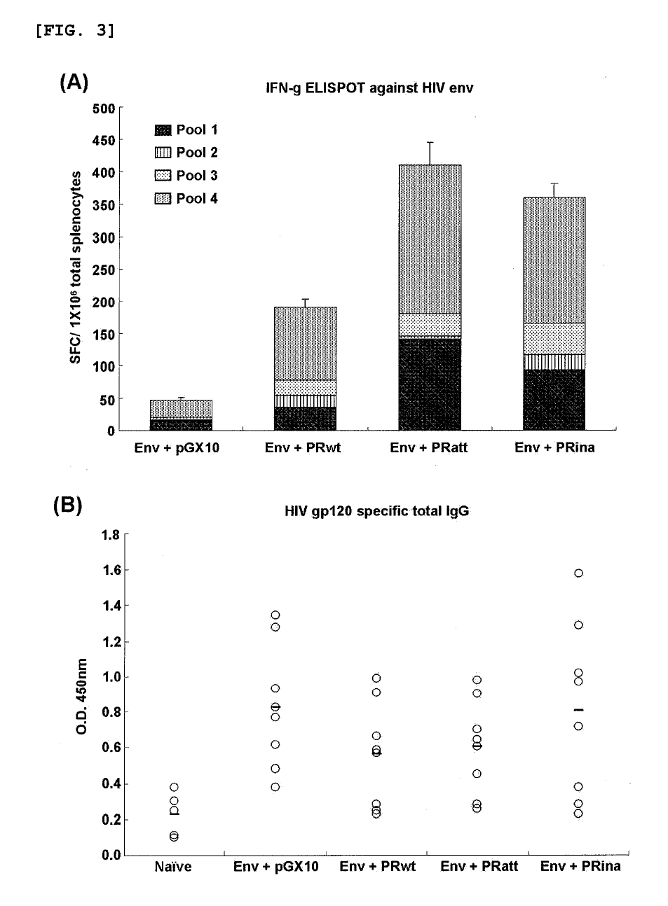

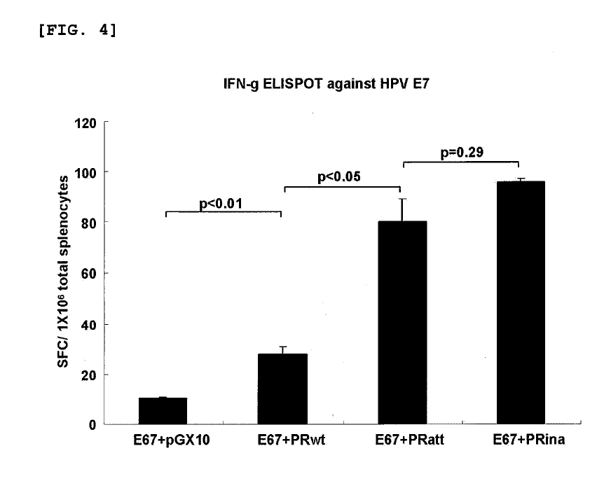

As a result, it was observed that co-immunization of antigen DNA with the wild type HIV-1 protease PRwt induced antigen-specific cell-mediated immune response that was slightly higher than a control group administered with antigen DNA and mock vector, whereas antigen-specific antibody responses were reduced (FIG. 3). It is suggested that this result is attributed to counter effects, namely, the cytotoxic effects of PRwt to enhance antigen targeting to dendritic cells and to reduce antigen expression. In contrast, it was observed that co-immunization with PRatt or PRina induced much higher antigen-specific cell-mediated immune response than PRwt (FIGS. 3 and 4), suggesting that this result is attributed to attenuation or inactivation in cytotoxic activity of HIV-1 protease, leading to minimizing inhibition of antigen expression or promoting its chaperone-like activity.

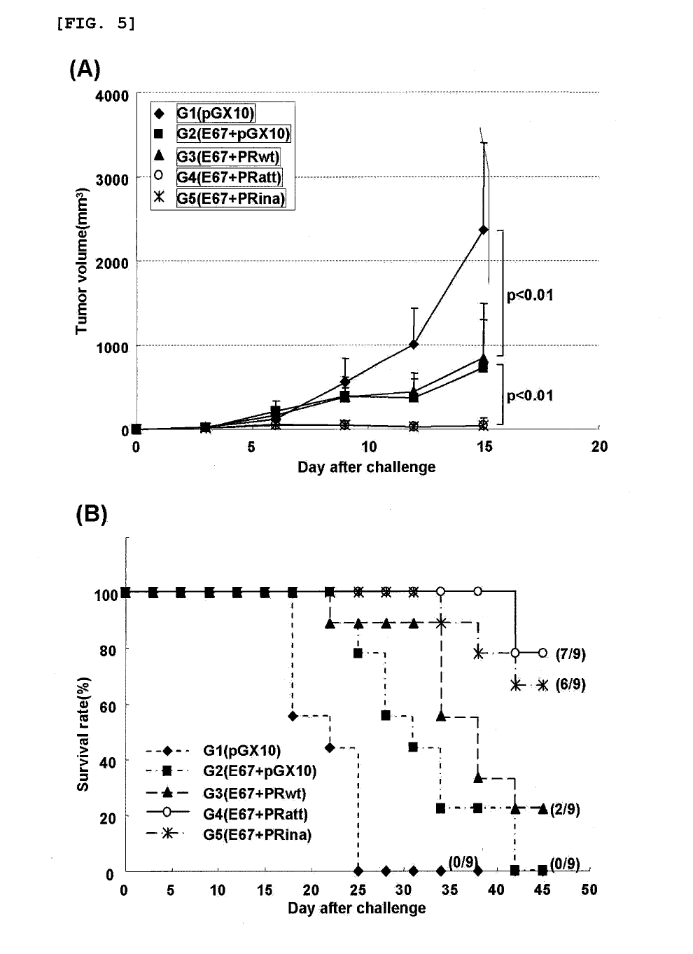

Such immune-enhancing effects of mutant HIV-1 protease on cell-mediated immune response were found to be exerted in DNA vaccines for the prevention of cervical cancer caused by HPV (human Papillomavirus), shown in the tumor inhibition test using HPV E6 and E7-expressing tumor cell line (FIG. 5). The experiments demonstrate that the adjuvant formulation comprising the mutant HIV-1 protease according to the present invention is able to enhance not only immune responses to the vaccines for the prevention or treatment of HIV-1 infection, but also immune responses to other vaccines for the prevention or treatment of a variety of diseases. Therefore, the vaccines that are able to enhance cell-mediated immune responses by co-administration with the adjuvant formulation comprising the mutant HIV-1 protease according to the present invention are exemplified by prophylactic and therapeutic vaccines for HIV-1, HPV, and hepatitis B or C, but are not limited thereto.

In accordance with still another embodiment, the present invention provides a vaccine composition that comprises a vaccine and the adjuvant formulation comprising the mutant HIV-1 protease or mutant SIV protease according to the present invention. The vaccine is preferably a DNA vaccine containing a nucleic acid encoding an antigen, and the mutant protease may be provided in a form of an expression vector or viral vector that includes a nucleic acid encoding the mutant protease. In this regard, the nucleic acid encoding an antigen and the nucleic acid encoding the mutant protease may be included in one expression vector or each of them may be included in the different vectors.

Further, the present invention provides a method for enhancing cell-mediated immune responses to a vaccine by administration of the vaccine together with the adjuvant formulation according to the present invention. In this connection, the adjuvant formulation and the vaccine may be given simultaneously or at any interval before or after each other. Preferably, they are administered simultaneously at the same site.

Meanwhile, in accordance with recent reports that certain immune deficiency states are associated with increased risk of cancer or the like, the adjuvant formulation according to the present invention can be used to enhance cell-mediated immune responses for cancer therapy by administration with an anticancer gene. Thus, the present invention provides an anticancer composition, comprising the adjuvant formulation according to the present invention and an anticancer gene for cancer therapy.

In Examples of the present invention, the experiments were performed using the mutant HIV-1 (Human Immunodeficiency Virus) protease. However, since there is 70% positivity and 51% identity in amino acid sequence of protease between SIV (simian immunodeficiency virus) and HIV-1, and their activities are similar to each other, it is postulated that the same results can be obtained by using the mutant SIV protease, as well as the mutant HIV-1 protease. Thus, those skilled in the art will appreciate that it is also included within the scope of the present invention.

### MODE FOR INVENTION

Hereinafter, the present invention will be described in more detail with reference to Examples. However, it will be apparent to those skilled in the art that these Examples are for the illustrative purpose only and the invention is not intended to be limited by these Examples.

### Example 1

### Construction of PRwt-Expressing DNA Vector

The codon-optimized nucleic acid (PRwt) that encodes the wild type HIV-1 protease having the nucleic acid sequence of SEQ ID NO: 1 was amplified by PCR using a pair of primers (5′ primer (SEQ ID NO: 2):

5′-GGTACCGCCACCATGGCTCCTCAGATAACACTTT-3′, introducing Asp718 site and Kozak sequence at 5′ end; and 3′ primer (SEQ ID NO: 3): 5′-TCTAGATTAGAAATTGAGAGTACAGCCGATCTGTGT-3′, introducing XbaI site at 3′ end) and a plasmid encoding the codon-optimized HIV-1 Pol as a template. The genes were digested with Asp718 and XbaI, and inserted into the pGX10 vector (Korean Patent Application No. 10-2006-0076619; PCT/KR2006/003181), so as to construct a plasmid expressing the gene, designated as pGX10-PRwt The enzyme active site (Asp-Thr-Gly) of PRwt prepared in Example 1 was changed to Asp-Ser-Gly and Ala-Thr-Gly by site directed mutagenesis, so as to construct pGX10-PRatt and pGX10-PRina which have the nucleic acid sequences of SEQ ID NOs: 4 and 5, respectively (FIG. 1).

### Example 2

### Confirmation of Different Proteolytic Activities of PRwt, PRatt and PRina

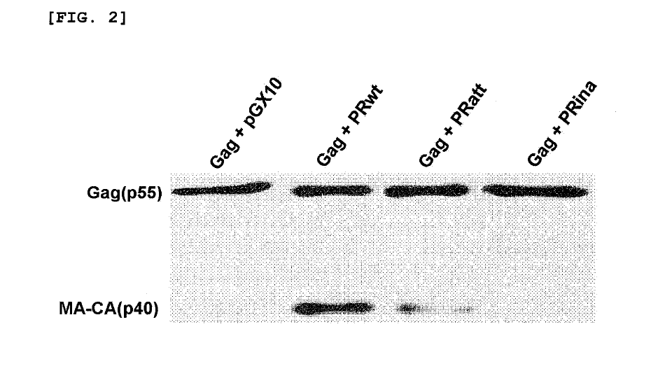

PRwt, PRatt and PRina prepared in Example 1 have different proteolytic activities from each other, and these activities are important for the induction of cell death by HIV-1 protease. It was known that PRatt has the proteolytic activity that is 5-10 fold lower than PRwt, and PRina completely loses the activity (Konvalinka, J., et al., J. Virol., 1995, 69:7180-7186, Babe, L. M., et al., Proc. Natl. Acad. Sci. USA, 1995, 92:10069-10073, Junker, U., et al., J. Virol., 1996, 70:7765-7772). In order to confirm this, HEK 293 cells (Human Embryonic Kidney 293 cell) were transfected with a plasmid (pGX10-gag) expressing HIV-1 Gag protein (MA-CA-NC-p6: Matrix-Capsid-Nucleocapsid-p6) that functions as a substrate of HIV-1 protease and a plasmid expressing PRwt, PRatt or PRina (each pGX10-PRwt, pGX10-PRatt, or pGX10-PRina) in the same amount. After 48 hrs, immunoblots were performed using cell lysates and anti-p24 (CA) mAb (NIBSC, Centre of AIDS reagent). As a result, protein processing of HIV-1 Gag poly-proteins was not observed in the group transfected with pGX10 vector and PRina, but MA-CA intermediate (p40) that is the processing product of HIV-1 Gag was observed in PRwt and PRatt-treated groups. The PRatt-treated group yielded a smaller amount of the MA-CA intermediate from HIV-1 Gag polyprotein, compared to the PRwt-treated group, indicating that the proteolytic activity of PRatt was more attenuated than PRwt (FIG. 2). The pGX10-Gag was prepared through codon optimization (Genscript) on the basis of consensus sequence of HIV-1 B subtype (Los Alamos database. http://www.hiv.lanlgov/content/index). The pGX10 fragment (3.6 kb) that was digested with Asp718 and Xba I restriction enzymes was ligated with the codon-optimized Gag gene having the same enzyme site at its both ends, so as to prepare pGX10-Gag.

### Example 3

### Immune-Enhancing Effects of PRwt, PRatt and PRina on Antigen-Specific Immune Responses in HIV Antigen Model

In order to confirm the immune-enhancing effects of PRwt, PRatt and PRina prepared in Example 1 on antigen-specific immune responses, female Balb/c mice were injected with 20 μg of pGX10-Env that is a DNA vaccine expressing HIV-1 envelope protein antigen (Env) and 20 μg of pGX10 vector, pGX10-PRwt, pGX10-PRatt and pGX10-PRina, respectively. Intramuscular injections were performed twice with a 3-week interval. 3 weeks after the last immunization, spleen cells were isolated from the mice. 1×10⁶ spleen cells were stimulated with HIV-1 Env peptide for 24 hrs, and IFN-γ ELISPOT assay was performed to analyze the number of IFN-γ-secreting T cell.

The pGX10-Env was prepared through codon optimization (Genscript) that substitutes TPA (tissue plasminogen activator) signal sequence for the intrinsic signal sequence capable of promoting extracellular secretion of antigen and removes the transmembrane region, on the basis of consensus sequence of HIV-1 B subtype (Los Alamos database. http://www.hiv.lanl.gov/content/index). The pGX10 fragment (3.6 kb) that was digested with Asp718 and Xba I restriction enzymes was ligated with the codon-optimized Env gene having the same enzyme site at its both ends, so as to prepare pGX10-Env.

As a result, it was observed that mice administered with PRwt and the antigen-expressing DNA vaccine exhibited higher antigen-specific T cell immune response than those administered with pGX10 vector and antigen. Meanwhile, it was observed that co-administration of PRatt or PRina together with the antigen-expressing DNA vaccine greatly enhanced antigen-specific cell-mediated immune response than administration of PRwt with antigen-expressing DNA vaccine (FIG. 3).

Unlike the effects of PRatt and PRina on the cell-mediated immune response, there was no significant difference in antigen-specific humoral immune response, as compared to the control group administered with pGX10 vector and antigen-expressing DNA vaccine, indicating that the immune-enhancing effect of the mutant HIV-1 protease is limited to cell-mediated immune response.

### Example 4

### Immune-Enhancing Effects of PRwt, PRatt and PRina on Antigen-Specific Immune Responses in HPV Antigen Model

In order to confirm the immune-enhancing effects of PRatt and PRina on antigen-specific immune responses in other mouse strains, female C57BL/6 mice were injected with 50 μg of pGX10-E67 antigen and 50 μg of pGX10 vector, pGX10-PRwt, pGX10-PRatt, or pGX10-PRina. Intramuscular injections were performed twice with a 3-week interval. 3 weeks after the last immunization, spleen cells were isolated from the mice. 1×10⁶ spleen cells were stimulated with HPV 16 E7 CD8⁺ T cell peptide for 24 hrs, and IFN-γ ELISPOT assay was performed to analyze the number of IFN-γ-secreting CD8+ T cell.

For pGX10-E67 preparation, PCR was performed using pcDNA3/E6 and pcDNA3/E7 genes provided by Dr. T. C. Wu (Johns Hopkins university) as a template and a pair of primers mentioned below.

E6 primer pair:
5′ primer
(SEQ ID NO: 6)
(5′ GGCCGAATTCATGCACCAAAAGAGAACTGCA 3′,
introducing EcoRI site)
and
3′ primer
(SEQ ID NO: 7)
(5′ CCGGGGATCCGCTTCCCAGCTGGGTTTCTCTACGTGTTCTTGA
3′, eliminating stop codon and introducing BamHI
site)
E7 primer pair:
5′ primer
(SEQ ID NO: 8)
(5′ GGCCGGATCCGGCAGCATGCATGGAGATACACCTACATTG 3′,
introducing BamHI site)
and
3′ primer
(SEQ ID NO: 9)
(5′ CCGGTCTAGATTATGGTTTCTGAGAACAGAT 3′,
introducing Xba I site)

The pSK (+) vector that was digested with EcoRI and BamHI restriction enzymes was ligated with E6 gene having the same enzyme site at its both ends, so as to prepare pSK (+)-E6. E7 fragment digested with BamHI and Xba I restriction enzymes was ligated with pSK (+)-E6 digested with the same restriction enzymes, so as to prepare pSK (+)-E67, and then inserted into pGX10 using EcoRI and Xba I restriction enzymes, resulting in pGX10-E67.

As a result, it was observed that co-administration of PRatt or PRina together with the antigen-expressing DNA vaccine greatly enhanced HPV E7 antigen-specific CD8 T cell immune response, like the results in HIV-1 Env antigen model, indicating that the mutant HIV-1 protease can be used to enhance cell-mediated immune responses to DNA vaccination, regardless of the type of antigen or the mouse model, and further that it can be effectively utilized as an adjuvant for DNA vaccines or therapeutic agents for the prevention or treatment of HIV, hepatitis B or C, and cancer, etc (FIG. 4).

### Example 5

### Immune-Enhancing Effects of PRwt, PRatt and PRina on Anticancer Efficacy

In order to confirm the effect of the mutant HIV-1 protease, PRatt and PRina on the prevention of cervical cancer, female C57BL/6 mice were injected with 50 μg of the antigen-expressing DNA vaccine pGX10-E67 and 50 μg of pGX10 vector, pGX10-PRwt, pGX10-PRatt, or pGX10-PRina. Intramuscular injections were performed twice with a 3-week interval. 3 weeks after the last immunization, the HPV16 E6 and E7-expressing tumor cell, TC-1 (5×10⁵ cells) was subcutaneously injected to the mouse, and tumor volume and survival rate were measured. As a result, it was observed that co-administration of PRatt or PRina together with the antigen-expressing DNA vaccine exhibited excellent anticancer effect on TC-1 tumor cells, compared to the control group, and survival rate was also remarkably increased (FIG. 5), suggesting that such anticancer effects are significantly correlated with the antigen-specific CD8 T cell immune response enhanced by the mutant HIV-1 protease.

### Example 6

### Relationship Between Immune-Enhancing Effects of PRwt, PRatt and PRina and CD4+ T Cell Help

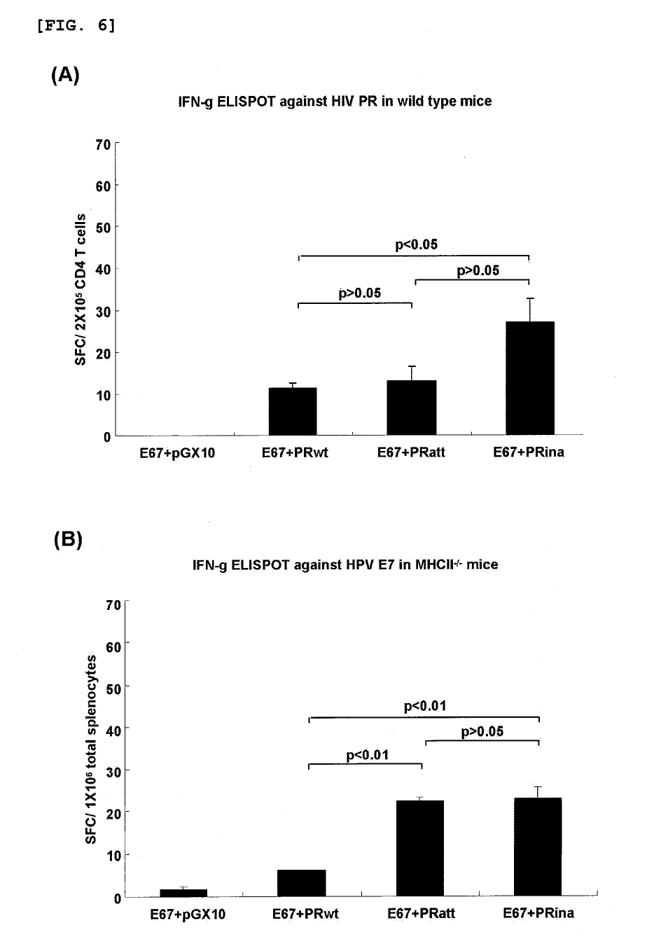

It was observed that CD4 T cell immune response to HIV-1 protease as an adjuvant for DNA vaccination was also enhanced by attenuating or inactivating the enzymatic activity of HIV-1 protease. This result suggests the possibility that immune-enhancing effects of the mutant HIV-1 protease are attributed to a bystander effect of CD4 T cell on HIV-1 protease. In order to confirm the possibility, immune-enhancing effects of the mutant HIV-1 protease were examined in the CD4 T cell-deficient, MHC class II^{-/-} mouse. Immunization was performed in the same manners as in <Example 4-5>. As a result, it was found that immune responses being similar to those in wild type mouse were induced (FIG. 6), indicating that immune-enhancing effects of the mutant HIV-1 protease are not attributed to a bystander effect of CD4 T cell on HIV-1 protease itself.

### INDUSTRIAL APPLICABILITY

The mutant HIV-1 protease or mutant SIV protease according to the present invention can be used to enhance cell-mediated immune responses to DNA vaccination, regardless of the type of antigen or the mouse model. Since cell-mediated immune responses are thought to be necessary to prevent or treat HIV, hepatitis B or C and cancer, the mutant protease can be effectively utilized as an adjuvant for DNA vaccines or therapeutic agents for the prevention or treatment of the diseases.

1. A vaccine adjuvant formulation comprising a mutant HIV-1 (Human immunodeficiency virus-1) protease or a mutant SIV (Simian immunodeficiency virus) protease which has inactivated or attenuated proteolytic activity, while retaining chaperone-like activity of HIV-1 protease or SIV protease.

2. The vaccine adjuvant formulation according to claim 1, wherein the mutant is prepared by modification of the enzyme active site, -Asp-Thr-Gly- in the amino acid sequence of wild type HIV-1 protease.

3. The vaccine adjuvant formulation according to claim 2, wherein the mutant is prepared by modification including substitution, deletion or insertion at the enzyme active site, -Asp-Thr-Gly- in the amino acid sequence of wild type HIV-1 protease.

4. The vaccine adjuvant formulation according to claim 3, wherein the mutant is prepared by substituting another amino acid for threonine (Thr) at the enzyme active site -Asp-Thr-Gly- in the amino acid sequence of wild type HIV-1 protease.

5. The vaccine adjuvant formulation according to claim 4, wherein the mutant is prepared by substituting serine (Ser) for threonine (Thr) at the enzyme active site -Asp-Thr-Gly- in the amino acid sequence of wild type HIV-1 protease.

6. The vaccine adjuvant formulation according to claim 3, wherein the mutant is prepared by substituting another amino acid for aspartic acid (Asp) at the enzyme active site -Asp-Thr-Gly- in the amino acid sequence of wild type HIV-1 protease.

7. The vaccine adjuvant formulation according to claim 6, wherein the mutant is prepared by substituting alanine (Ala) for aspartic acid (Asp) at the enzyme active site -Asp-Thr-Gly- in the amino acid sequence of wild type HIV-1 protease.

8. The vaccine adjuvant formulation according to claim 1, wherein the mutant HIV-1 protease is expressed by a codon-optimized nucleic acid sequence to increase its expression in mammalian cells.

9. An adjuvant formulation, comprising a nucleic acid encoding the mutant HIV-1 protease according to claim 1.

10. The adjuvant formulation according to claim 9, wherein the nucleic acid has a base sequence of SEQ ID NO: 4 or SEQ ID NO: 5.

11. The adjuvant formulation according to claim 10, wherein the nucleic acid is included in an expression vector.

12. The adjuvant formulation according to claim 11, wherein the expression vector is the pGX10 vector shown in FIG. 1.

13. The adjuvant formulation according to claim 10, wherein the nucleic acid is included in a viral vector.

14. The adjuvant formulation according to claim 13, wherein the viral vector is a vector derived from adenovirus, lentivirus, retrovirus, adeno-associated virus, vaccinia virus, or alphavirus.

15. A vaccine composition, comprising the vaccine adjuvant formulation according to claim 1 and an antigen.

16. The vaccine composition according to claim 15, wherein the antigen is a DNA vaccine consisting of a nucleic acid encoding an antigen.

17. The vaccine composition according to claim 16, wherein the nucleic acid encoding an antigen and the nucleic acid encoding the mutant HIV-1 protease that is included in the adjuvant formulation are included in one expression vector.

18. The vaccine composition according to claim 16, wherein the nucleic acid encoding an antigen and the nucleic acid encoding the mutant HIV-1 protease that is included in the adjuvant formulation are included in different expression vectors.

19. The vaccine composition according to claim 15, wherein the vaccine composition is a prophylactic and therapeutic vaccine for HW-1, HPV, and hepatitis B or C.

20. A method for enhancing cell-mediated immune responses to an antigen by administration of the adjuvant formulation according to claim 1 together with the antigen.

21. A method for treating cancer by administration of the adjuvant formulation according to claim 1 together with an anticancer gene.

22. An anticancer composition, comprising the adjuvant formulation according to claim 1 and an anticancer gene.

23. Use of a mutant HIV-1 (Human immunodeficiency virus-1) protease or mutant SW (Simian immunodeficiency virus) protease as a vaccine adjuvant formulation, wherein the mutant has inactivated or attenuated proteolytic activity, while retaining chaperone-like activity of HIV-1 protease or SIV protease.

24. The use according to claim 23, wherein the mutant is prepared by modification of the enzyme active site, -Asp-Thr-Gly- in the amino acid sequence of wild type HW-1 protease.

25. The use according to claim 24, wherein the mutant is prepared by modification including substitution, deletion or insertion at the enzyme active site, -Asp-Thr-Gly- in the amino acid sequence of wild type HIV-1 protease.

26. The use according to claim 25, wherein the mutant is prepared by substituting another amino acid for threonine (Thr) at the enzyme active site -Asp-Thr-Gly- in the amino acid sequence of wild type HIV-1 protease.

27. The use according to claim 26, wherein the mutant is prepared by substituting serine (Ser) for threonine (Thr) at the enzyme active site -Asp-Thr-Gly- in the amino acid sequence of wild type HIV-1 protease.

28. The use according to claim 25, wherein the mutant is prepared by substituting another amino acid for aspartic acid (Asp) at the enzyme active site -Asp-Thr-Gly- in the amino acid sequence of wild type HIV-1 protease.

29. The use according to claim 28, wherein the mutant is prepared by substituting alanine (Ala) for aspartic acid (Asp) at the enzyme active site -Asp-Thr-Gly- in the amino acid sequence of wild type HIV-1 protease.

30. The use according to claim 23, wherein the mutant HIV-1 protease is expressed by a codon-optimized nucleic acid sequence to increase its expression in mammalian cells.

31. Use of a nucleic acid encoding the mutant HIV-1 protease according to claim 23 as an adjuvant.

32. The use according to claim 31, wherein the nucleic acid has a base sequence of SEQ ID NO: 4 or SEQ ID NO: 5.


SEQUENCE LISTING (12 sequences)

SEQ ID NO: 1 - DNA, 306 bp, Artificial Sequence
  Description of Artificial Sequence Synthetic polynucleotide for codon-optimized wild type human HIV-1 protease (PRwt)
atggctcctcagataacactttggcagagacccctggtcaccataaagattggaggtcagctcaaggaagccttgctggacactggcgcagatgatacagtgctggaggaaatgaacctgcccggccggtggaagcctaaaatgatcggaggtattggcggctttatcaaggtgcgccagtacgatcaaattctgatcgaaatatgcggacataaagccatcggcacagttctggtgggaccaacacctgtgaatatcattggacgcaacctgctgacacagatcggctgtactctcaatttctaa

SEQ ID NO: 2 - DNA, 34 bp, Artificial Sequence
  Description of Artificial Sequence Synthetic 5′ primer for codon-optimized wild type HIV-1 protease
ggtaccgccaccatggctcctcagataacacttt

SEQ ID NO: 3 - DNA, 36 bp, Artificial Sequence
  Description of Artificial Sequence Synthetic 3′ primer for codon optimized wild type HIV-1 protease
tctagattagaaattgagagtacagccgatctgtgt

SEQ ID NO: 4 - DNA, 306 bp, Artificial Sequence
  Description of Artificial Sequence Synthetic polynucleotide for codon-optimized attenuated HIV-1 protease (PRatt)
atggctcctcagataacactttggcagagacccctggtcaccataaagattggaggtcagctcaaggaagccttgctggacagtggcgcagatgatacagtgctggaggaaatgaacctgcccggccggtggaagcctaaaatgatcggaggtattggcggctttatcaaggtgcgccagtacgatcaaattctgatcgaaatatgcggacataaagccatcggcacagttctggtgggaccaacacctgtgaatatcattggacgcaacctgctgacacagatcggctgtactctcaatttctaa

SEQ ID NO: 5 - DNA, 306 bp, Artificial Sequence
  Description of Artificial Sequence Synthetic polynucleotide for codon-optimized inactivated HIV-1 protease (PRina)
atggctcctcagataacactttggcagagacccctggtcaccataaagattggaggtcagctcaaggaagccttgctggctactggcgcagatgatacagtgctggaggaaatgaacctgcccggccggtggaagcctaaaatgatcggaggtattggcggctttatcaaggtgcgccagtacgatcaaattctgatcgaaatatgcggacataaagccatcggcacagttctggtgggaccaacacctgtgaatatcattggacgcaacctgctgacacagatcggctgtactctcaatttctaa

SEQ ID NO: 6 - DNA, 31 bp, Artificial Sequence
  Description of Artificial Sequence Synthetic 5′ primer for HPV E6 gene
ggccgaattcatgcaccaaaagagaactgca

SEQ ID NO: 7 - DNA, 43 bp, Artificial Sequence
  Description of Artificial Sequence Synthetic 3′ primer for HPV E6 gene
ccggggatccgcttcccagctgggtttctctacgtgttcttga

SEQ ID NO: 8 - DNA, 40 bp, Artificial Sequence
  Description of Artificial Sequence Synthetic 5′ primer for HPV E7 gene
ggccggatccggcagcatgcatggagatacacctacattg

SEQ ID NO: 9 - DNA, 31 bp, Artificial Sequence
  Description of Artificial Sequence Synthetic 3′ primer for HPV E7 gene
ccggtctagattatggtttctgagaacagat

SEQ ID NO: 10 - protein, 101 aa, Human immunodeficiency virus 1
  Wild type HIV-1 protease (PRwt)
MAPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNF

SEQ ID NO: 11 - protein, 101 aa, Artificial Sequence
  Description of Artificial Sequence Synthetic polypeptide for attenuated HIV-1 protease (PRatt)
MAPQITLWQRPLVTIKIGGQLKEALLDSGADDTVLEEMNLPGRWKPKMIGGIGGFIKVRQYDQILIEICGHKAIGTVLVGPTPVNIIGRNLLTQIGCTLNF

SEQ ID NO: 12 - protein, 101 aa, Artificial Sequence
  Description of Artificial Sequence Synthetic polypeptide for inactivated HIV-1 protease (PRina)
MAPQITLWQRPLVTIKIGGQLKEALLATGADDTVLEEMNLPGRWKPKMIGGIGGFIKVRQYDQILIEICGHKAIGTVLVGPTPVNIIGRNLLTQIGCTLNF